SEQUENCE LISTING (2 sequences)

SEQ ID NO: 1 - DNA, 20 bp, Artificial
  Forward primer sequence
ccgggaacggtgcattggaa

SEQ ID NO: 2 - DNA, 20 bp, Artificial
  Reverse primer sequence
atcggcctcaggaagatcgc

(19) United States
(12) Patent Application Publication
(10) Pub. No.: US 2006/0036209 A1
(43) Pub. Date: Feb. 16, 2006

(54) System and method for transdermal delivery

(76) Inventors: Janardhanan Subramony, Santa Clara, CA (US); Georg Widera, Palo Alto, CA (US); Joseph B. Phipps, Sunnyvale, CA (US); Michel Cormier, Mountain View, CA (US)

Correspondence Address: Ralph C.Francis;Francis Law Group, 1808 Santa Clara Ave, Alameda, CA, 94501, US

(21) Appl. No.: 10/978,806

(22) Filed: Nov. 1, 2004

Related U.S. Application Data
(60) Provisional application No. 60/520,043, filed on Nov. 13, 2003.

(51) Int. Cl. A61N 1/30

(52) U.S. Cl. 604/20

(57) ABSTRACT

A system and method for transdermally delivering a biologically active agent comprising one or more electrodes having stratum corneum-piercing projections and a circuit that delivers an electrical signal to the electrodes to electroporate a cell membrane. Preferably, the system is configured to generate homogeneous electrical fields and, more preferably, to generate spherically or semispherically symmetrical electric fields. Methods of the invention include applying a first electric signal to facilitate transdermal transport of the agent and applying a second electric signal to facilitate intracellular transport of the agent.

### CROSS-REFERENCE TO RELATED APPLICATIONS

This application claims the benefit of U.S. Provisional Application No. 60/520,043, filed Nov. 13, 2003.

### FIELD OF THE PRESENT INVENTION

The present invention relates generally to transdermal delivery systems and methods. More particularly, the invention relates to a percutaneous and intracellular delivery system utilizing electric potential to facilitate the movement of a substance.

### BACKGROUND OF THE INVENTION

Active agents (or drugs) are most conventionally administered either orally or by injection. Unfortunately, many active agents are completely ineffective or have radically reduced efficacy when orally administered since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure that sometimes results in poor patient compliance.

The word “transdermal” is used herein as a generic term referring to passage of an agent across the skin layers. The word “transdermal” refers to delivery of an agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle.

Hence, in principle, transdermal delivery provides for a method of administering active agents that would otherwise need to be delivered orally or via hypodermic injection or intravenous infusion. Transdermal agent delivery offers improvements in these areas. Transdermal delivery, when compared to oral delivery, avoids the harsh environment of the digestive tract, bypasses gastrointestinal agent metabolism, reduces first-pass effects, and avoids the possible deactivation by digestive and liver enzymes. Likewise, the digestive tract is not subjected to the active agent during transdermal administration since many agents, such as aspirin, have an adverse effect on the digestive tract. Transdermal delivery also offers advantages over the more invasive hypodermic or intravenous agent delivery options. Specifically, no significant cutting or penetration of the skin is necessary, such as cutting with a surgical knife or piercing the skin with a hypodermic needle. This minimizes the risk of infection and pain.

While active agents do diffuse across both the stratum corneum and the epidermis, the rate of diffusion through the highly ordered lipid bilayers of the stratum corneum is often the limiting step. Thus, in many instances, the rate of delivery or flux of many agents, particularly macromolecules, via the passive transdermal route is too limited to be therapeutically effective.

To improve upon the transdermal flux of passive diffusion, external energy sources, such as electricity (e.g., iontophoresis and electroporation) and ultrasound (e.g., phonophoresis) can be employed to assist transport of an active agent.

Electrotransport transdermal delivery devices generally employ two electrodes that are positioned in intimate contact with some portion of the body, typically the skin. A first electrode, called the active or donor electrode, is used to deliver the therapeutic agent into the body. The second electrode, called the counter or return electrode, closes an electrical circuit with the first electrode through the body. A source of electrical energy, such as a battery, supplies electric current to the body through the electrodes. For example, if the therapeutic agent to be delivered into the body is a positively charged cation, the anode is the active electrode and the cathode is the counter electrode required to complete the circuit. If the therapeutic agent to be delivered is a negatively charged anion, the cathode is the donor electrode and the anode is the counter electrode.

A widely used electrotransport process, electromigration (also called iontophoresis), involves the electrically induced transport of charged ions (e.g., drug ions) through a body surface. Another type of electrotransport, called electroosmosis, involves the trans-body surface (e.g., transdermal) flow of a liquid under the influence of the applied electric field. Still another type of electrotransport process, called electroporation, involves forming transiently existing pores in a biological membrane by applying high voltage pulses.

Other attempts to improve transdermal flux have utilized small skin piercing elements to physically penetrate the stratum corneum. Examples of these approaches are disclosed in European Patent EP 0 407063A1, U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365.

There have also been attempts in the prior art to combine mechanical penetration of the skin with iontophoresis to effect transdermal delivery. For example, U.S. Pat. No. 6,591,133 discloses a combination of needles and electric potential to deliver material through a patient's skin. The noted system employs one or more needles, which are used to pierce the stratum corneum and can also be used as electrodes. Similarly, U.S. Pat. No. 6,256,533, discloses the use of microneedles together with iontophoresis for transdermal delivery and extraction. These prior art systems are designed to move material across the skin of a patient, but are not directed at the delivery of material into cells, nor do they provide means for increasing the symmetry and uniformity of the applied electrical field.

It is therefore an object of the present invention to provide a transdermal agent delivery system and method that provides an improvement over prior art agent delivery systems.

Accordingly, it is an object of the present invention to provide a transdermal agent delivery system and method having an electrical field with improved homogeneity and symmetry to deliver a biologically active agent.

It is another object of the invention to provide a system and method to electroporate cell membranes and provide intracellular delivery of a biologically active agent using an applied electric field.

It a further object of the present invention to provide a system and method to improve transdermal delivery of a biologically active agent using an applied electric field.

Yet another object of the present invention is to provide a transdermal agent delivery system that is configured to produce a spherically or semispherically symmetric electric field.

It is another object of the present invention to provide a transdermal agent delivery system that enhances electric field densities.

### SUMMARY OF THE INVENTION

In accordance with the above objects and those that will be mentioned and will become apparent below, the system for transdermally delivering a biologically active agent in accordance with this invention comprises a microprojection member adapted to provide an electrical field capable of electroporating cellular membranes to facilitate intracellular transport of the agent.

In one embodiment of the invention, the transdermal delivery system comprises a first electrode having top and bottom surfaces and a plurality of stratum corneum-piercing microprojections that protrude from the bottom surface of the first electrode, a second electrode, a biologically active agent source associated with the first electrode containing a biologically active agent and a circuit adapted to deliver a first electrical signal to the first and second electrodes capable of electroporating a cell membrane. Accordingly, applying the first electrical signal facilitates intracellular delivery of the biologically active agent.

In such embodiments, the first electrical signal is preferably configured to generate electric field densities in the range of approximately 100 V/cm to 5,000 V/cm.

Preferably, the circuit is also adapted to deliver a second electrical signal to the electrodes, prior to the first, that facilitates transdermal delivery of the biologically active agent.

Also preferably, the second electrode has top and bottom surfaces and a plurality of stratum corneum-piercing microprojections that protrude from the bottom surface of the electrode. Preferably, the first and second electrodes generate a substantially homogenous electrical field.

In one aspect of the invention, the first and second electrodes comprise a first integral microprojection member.

In one embodiment, the first electrode and the second electrode comprise zones of the microprojection member, separated by an insulator. Preferably, the first electrode comprises a circular zone of the microprojection member and the second electrode comprises a circumferential zone around the circular zone.

More preferably, delivery of the first electrical signal generates a spherically symmetrical electric field and a substantially homogenous electrical field. In the noted embodiments, the first electrode and the second electrode can comprises a parallel plate capacitor geometry around a circumference of the microprojection member.

In an alternative embodiment, the first electrode and the second electrode comprise alternating rows of the stratum corneum-piercing microprojections separated by an insulator.

In yet another embodiment of the invention, the first electrode and the second electrode comprise separate microprojection members. The second electrode is preferably positioned relative to the first electrode to generate a semispherically symmetrical electrical field.

Another aspect of the invention comprises an insulating coating disposed on the first microprojection member configured to maximize electric field densities to electroporate cells. Preferably, the insulating coating is disposed on the bottom surface of the electrodes and on a portion of the stratum corneum-piercing microprojections. In such embodiments, each of the plurality of stratum corneum-piercing microprotrusions comprises a tip and the insulating coating is preferably not disposed on the tip.

In certain embodiments of the invention, one or more of the microprojections of the first or second electrode comprise a barb configured to anchor the microprojection member to a patient's skin.

In another embodiment, the microprojections of the invention have a length less than approximately 1000 microns, and more preferably, a length less than approximately 500 microns. The stratum corneum-piercing microprotrusions of the invention can also have a thickness in the range of approximately 5-50 microns.

In certain embodiments of the invention, the biologically active agent comprises an immunologically active agent, such as a vaccine or antigen. Exemplary vaccines include viruses and bacteria, protein-based vaccines, polysaccharide-based vaccine, and nucleic acid-based vaccines. Futher details regarding delivery of vaccines and other immunologically active agents is found in Co-Pending Application Ser. No. 60/516,184, and Serial No. ______, filed ______. [Attorney Docket No. ALZ5085NP], which are hereby incorporated in their entirety by reference.

In other embodiments of the invention, the biologically active agent comprises an agent active in one of the major therapeutic areas including, but not limited to: anti-infectives, such as antibiotics and antiviral agents; analgesics, including fentanyl, sufentanil, remifentanil, buprenorphine and analgesic combinations; anesthetics; anorexics; antiarthritics; antiasthmatic agents such as terbutaline; anticonvulsants; antidepressants; antidiabetic agents; antidiarrheals; antihistamines; anti-inflammatory agents; antimigraine preparations; antimotion sickness preparations such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists such as dobutamine and ritodrine; antiarrythmics; antihypertensives such as atenolol; ACE inhibitors such as ranitidine; diuretics; vasodilators, including general, coronary, peripheral, and cerebral; central nervous system stimulants; cough and cold preparations; decongestants; diagnostics; hormones such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable agents include vasoconstrictors, anti-healing agents and pathway patency modulators.

In a preferred embodiment of the invention, the biologically active agent source comprises a biocompatible coating that is disposed on the microprojection member. Details regarding suitable coating formulations are found in Co-Pending Application Ser. No. 60/516,184, Serial No. ______, filed ______ [Attorney Docket No. ALZ5049] and Serial No. ______, filed ______ [Attorney Docket No. ALZ5085NP], which are hereby incorporated in their entirety by reference.

As described in greater detail below, particularly preferred compounds that can be incorporated in the biocompatible coatings of the invention include a surfactant, an amphiphilic polymer, a hydrophilic polymer, a biocompatible carrier, a stabilizing agent, a vasoconstrictor, and/or a pathway patency modulator.

In other embodiments of the invention, the biologically active agent source can comprise an agent reservoir disposed adjacent the donor electrode that is adapted to contain a hydrogel formulation. Further details regarding suitable hydrogel formulations can be found in Co-Pending Application No. 60/514,387, filed Oct. 24, 2003, which is incorporated by reference herein in its entirety.

As described in greater detail below, particularly preferred compounds that can be incorporated in the hydrogel formulations of the invention include a macromolecular polymer network, a surfactant, an amphiphilic polymer, a vasoconstrictor, and/or a pathway patency modulator.

According to the invention, the biologically active agent to be delivered can be contained in the hydrogel formulation disposed in a gel pack reservoir, contained in a biocompatible coating that is disposed on the microprojection member or contained in both the hydrogel formulation and the biocompatible coating. Furthermore, embodiments that comprise the biologically active agent in a coating can also employ a hydrogel reservoir to hydrate and dissolve the coating.

The invention also comprises a method for delivering a biologically active agent comprising the steps of providing a transdermal delivery system that comprises a first electrode having top and bottom surfaces and a plurality of stratum corneum-piercing microprojections that protrude from the bottom surface of the first electrode, a second electrode, a biologically active agent source associated with the first electrode containing a biologically active agent and a circuit adapted to deliver a first electrical signal to the first and second electrodes capable of electroporating a cell membrane; and delivering a first electrical signal to the first electrode and the second electrode configured to facilitate intracellular transport of the biologically active agent. Preferably, such methods further comprise the step of delivering a second electrical signal to the first electrode and the second electrode, prior to the first electrical signal, that facilitates transdermal transfer of the biologically active agent. The first electrical signal is preferably configured to generate electric field densities in the range of approximately 100 V/cm to 5,000 V/cm.

Methods of the invention also preferably comprise the step of repeatedly delivering the first electrical signal.

Also preferably, the second electrode has top and bottom surfaces and a plurality of stratum corneum-piercing microprojections that protrude from the bottom surface.

Methods of the invention preferably comprise the step of delivering a first electrical signal to generate a substantially homogenous electric field.

In one embodiment, the invention comprises providing the system wherein the first and second electrodes comprise a first microprojection member.

Preferably, the method comprises providing the system wherein the first electrode comprises a circular zone of the microprojection member and the second electrode comprises a circumferential zone around the circular zone. Accordingly, delivery of the first electrical signal generates a spherically symmetrical electric field.

Alternatively, the first and second electrodes comprise alternating rows of the stratum corneum-piercing microprojections on the first microprojection member, wherein the alternating rows are separated by an insulator.

In another embodiment of the invention, the method comprises providing the system wherein the first electrode comprises a first microprojection member and the second electrode comprises a second microprojection member. Preferably, delivering the first electrical signal generates a substantially homogenous electrical field. Also preferably, the first and second microprojection members are positioned so that delivering the first electrical signal generates a semispherically symmetrical electrical field.

Other methods of the invention further comprise the step of disposing an insulating coating on the first microprojection member that is configured to maximize electric field densities to electroporate cells. Preferably, the step of disposing an insulating coating on the first microprojection member comprises leaving tips of the stratum corneum-piercing microprojections uncoated.

In yet another embodiment of the invention, the method comprises delivering a first electrical signal to the electrodes adapted to transdermally deliver the biologically active agent, delivering a second electrical signal adapted to electroporate a cell membrane and subsequently delivering a third electrical signal to the electrodes adapted to transport the biologically active agent across the cell membrane.

In one preferred embodiment of the invention, the step of delivering a biologically active agent comprises delivering an immunologically active agent, such as viruses, bacteria, protein-based vaccines, polysaccharide-based vaccines, nucleic acid-based vaccines, proteins, polysaccharide conjugates, oligosaccharides, antigenic agents and lipoproteins.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an exploded perspective view of one embodiment of the system of the invention;

FIG. 2 is a sectional side view of another embodiment of the invention;

FIG. 3 is perspective view with detail of a microprojection member of the invention and an exemplary applicator;

FIG. 4 is a perspective view of a microprojection member, according to the invention;

FIG. 5 is a schematic view of one embodiment of a system for transdermally delivering a biologically active agent, according to the invention;

FIG. 6 is a detail view of a portion of the microprojection member of the system shown in FIG. 5;

FIG. 7 is a detail schematic view showing a portion of the system shown in FIG. 5;

FIG. 8 is a schematic view of the dipolar charge distribution profile that can be generated using the embodiment shown in FIG. 5;

FIG. 9 is a schematic view of the electric field generated by the microprojection member shown in FIG. 5;

FIG. 10 is a schematic view of the electric field generated by another embodiment of the invention;

FIG. 11 is a partial perspective view of a microprojection member representing one embodiment of the invention; and

FIG. 12 is a partial perspective view of a microprojection member representing another embodiment of the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy. The term “transdermal flux”, as used herein, means the rate of transdermal delivery.

The term “biologically active agent”, as used herein, refers to a composition of matter or mixture containing a drug which is pharmacologically effective when administered in a therapeutically effective amount. The term “agent” is also intended to have its broadest interpretation and is used to include any therapeutic agent or drug. The terms “drug”, “therapeutic agent” and “biologically active agent” are used interchangeably to refer to any therapeutically active substance that is delivered to a living organism to produce a desired, usually beneficial, effect.

Particularly preferred biologically active agents include, without limitation, immunologically active agents, for example viruses, bacteria, protein-based vaccines, polysaccharide-based vaccines, proteins, polysaccharide conjugates, oligosaccharides, lipoproteins, single-stranded and double-stranded nucleic acids, polynucleotide constructs for gene therapy, RNA molecules, such as, for example, mRNA, antisense oligonucleotides, ribozymes, and siRNA (RNAI) molecules, chromosomes, conventional vaccines, DNA vaccines, immunogenic materials, antigenic agents and vaccine adjuvants. Specific examples of vaccine delivery can be found in Co-Pending Application Ser. No. 60/516,184 and Ser. No. ______, filed ______. [Attorney Docket No. ALZ5085NP], which are hereby incorporated in their entirety by reference.

Particularly with regard to protein-based vaccines and DNA vaccines, electrotransport preferably provides in vivo intracellular delivery of the vaccine. In the case of protein-based vaccines, this delivery into skin-presenting cells leads to cellular loading of the protein-based vaccine epitopes onto class I MHC/HLA presentation molecules in addition to class II MHC/HLA presentation molecules in a subject. Preferably, a cellular and humoral response is produced.

With respect to DNA vaccines, delivery of the DNA-based vaccine into skin-presenting cells leads to cellular expression of the vaccine antigen encoded by the DNA vaccine and loading of the vaccine epitopes onto class I MHC/HLA presentation molecules in addition to class II MHC/HLA presentation molecules in a subject. Also preferably, a cellular and humoral response in produced in the subject. Alternatively, only a cellular response is produced.

Suitable immunologically active agents include, without limitation, antigens in the form of proteins, polysaccharide conjugates, oligosaccharides, and lipoproteins. These subunit vaccines include Bordetella pertussis (recombinant PT accince-acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired.

In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)—N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

As will be appreciated by one having ordinary skill in the art, with few exceptions, alum-adjuvanted vaccine formulations typically lose potency upon freezing and drying. To preserve the potency and/or immunogenicity of the alum-adsorbed vaccine formulations of the invention, the noted formulations can be further processed as disclosed in Provisional application Ser. No. ______ [Attorney Docket No. ALZ5156PSP1, filed Sep. 28, 2004]; which is expressly incorporated by reference herein in its entirety.

The biologically active agent can also comprise an agent active in one of the major therapeutic areas including, but not limited to: anti-infectives such as antibiotics and antiviral agents; analgesics, including fentanyl, sufentanil, remifentanil, buprenorphine and analgesic combinations; anesthetics; anorexics; antiarthritics; antiasthmatic agents such as terbutaline; anticonvulsants; antidepressants; antidiabetic agents; antidiarrheals; antihistamines; anti-inflammatory agents; antimigraine preparations; antimotion sickness preparations such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists such as dobutamine and ritodrine; antiarrythmics; antihypertensives such as atenolol; ACE inhibitors such as ranitidine; diuretics; vasodilators, including general, coronary, peripheral, and cerebral; central nervous system stimulants; cough and cold preparations; decongestants; diagnostics; hormones such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable agents include vasoconstrictors, anti-healing agents and pathway patency modulators.

Further specific examples of agents include, without limitation, growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

The noted biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

It is to be understood that more than one biologically active agent may be incorporated into the agents source, reservoirs, and/or coatings of this invention, and that the use of the term “active agent” in no way excludes the use of two or more such active agents or drugs.

The term “biologically effective amount” or “biologically effective rate” shall be used when the biologically active agent is a pharmaceutically active agent and refers to the amount or rate of the pharmacologically active agent needed to effect the desired therapeutic, often beneficial, result. The amount of active agent employed in the hydrogel formulations and coatings of the invention will be that amount necessary to deliver a therapeutically effective amount of the active agent to achieve the desired therapeutic result.

In practice, this will vary widely depending upon the particular pharmacologically active agent being delivered, the site of delivery, the severity of the condition being treated, the desired therapeutic effect and the dissolution and release kinetics for delivery of the active agent from the coating into skin tissues.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections typically have a width and thickness of about 5 to 50 microns. The microprojections may be formed in different shapes, such as needles, hollow needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 4. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “electrotransport”, as used herein, refers generally to the delivery or extraction of a therapeutic agent (charged, uncharged, or mixtures thereof) through a body surface (such as skin, mucous membrane, or nails) wherein the delivery or extraction is at least partially induced or aided by the application of an electric potential. The electrotransport process has been found to be useful in the transdermal administration of many drugs including lidocaine, hydrocortisone, fluoride, penicillin, and dexamethasone. A common use of electrotransport is in diagnosing cystic fibrosis by delivering pilocarpine iontophoretically.

A widely used electrotransport process, electromigration (also called iontophoresis), involves the electrically induced transport of charged ions (e.g., agent ions) through a body surface. Another type of electrotransport, called electroosmosis, involves the trans-body surface (e.g., transdermal) flow of a liquid under the influence of the applied electric field.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported.

The term “electroporation”, as used herein, generally recognizes that exposing cells to strong electric fields for brief periods of time can temporarily destabilize the cell membranes. This effect has been described as a dielectric breakdown due to an induced transmembrane potential, and may also be referred to as “electropermeabilization.” Preferably, the permeabilized state of the cell membrane is transitory. Typically, cells remain in a destabilized state on the order of minutes after electrical treatment ceases.

As indicated above, the present invention comprises a system and method for transdermally delivering a biologically active agent to a patient. The system generally includes an active electrode and a donor electrode and electric circuitry for supplying electrical signals to the electrodes. For agent delivery, a source of biologically active agents is provided adjacent at least one of the electrodes. One or both electrodes comprise a microprojection member having a plurality of stratum corneum-piercing microprojections extending therefrom.

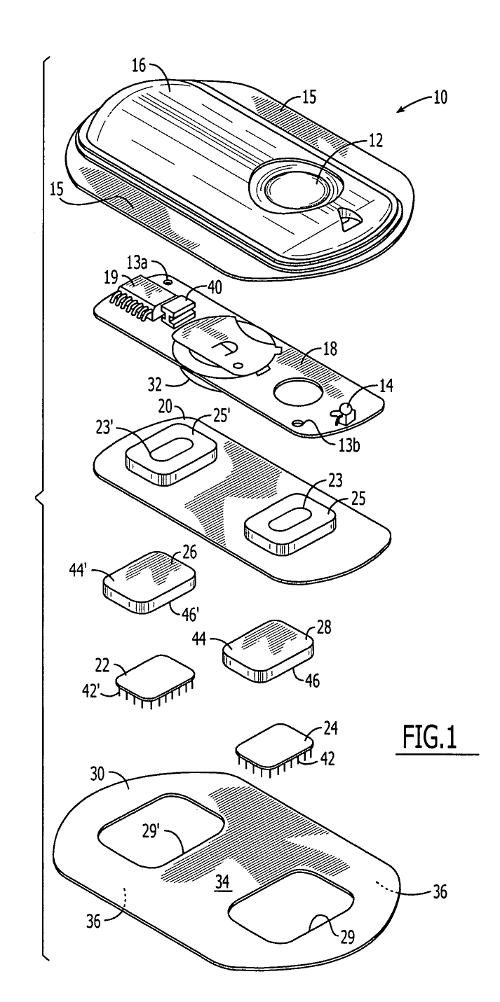

Reference is now made to FIG. 1, which depicts an exemplary electrotransport device that can be used in accordance with the present invention. FIG. 1 shows a perspective exploded view of an electrotransport device 10 having an activation switch in the form of a push button switch 12 and a display in the form of a light emitting diode (LED) 14. Device 10 comprises an upper housing 16, a circuit board assembly 18, a lower housing 20, anode electrode 22, cathode electrode 24, anode reservoir 26, cathode reservoir 28 and skin-compatible adhesive 30. Upper housing 16 has lateral wings 15 that assist in holding device 10 on a patient's skin. Upper housing 16 is preferably composed of an injection moldable elastomer (e.g., ethylene vinyl acetate). Printed circuit board assembly 18 comprises an integrated circuit 19 coupled to discrete electrical components 40 and battery 32. Circuit board assembly 18 is attached to housing 16 by posts (not shown in FIG. 1) passing through openings 13a and 13b, the ends of the posts being heated/melted in order to heat stake the circuit board assembly 18 to the housing 16. Lower housing 20 is attached to the upper housing 16 by means of adhesive 30, the upper surface 34 of adhesive 30 being adhered to both lower housing 20 and upper housing 16 including the bottom surfaces of wings 15.

Shown (partially) on the underside of circuit board assembly 18 is a battery 32, preferably a button cell battery and most preferably a lithium cell. Other types of batteries may also be employed to power device 10.

The circuit outputs (not shown in FIG. 1) of the circuit board assembly 18 make electrical contact with the top sides 44′, 44 of reservoirs 26 and 28 through openings 23, 23′ in the depressions 25, 25′ formed in lower housing. Electrodes 22 and 24, in turn, are in direct mechanical and electrical contact with the bottom sides 46′, 46 of reservoirs 26 and 28. Electrodes 22 and 24 comprise microprojection array members, each having a plurality of microprojections 42′, 42 (not shown to scale) and openings to allow passage of agent or salt from reservoirs 26 and 28 (as described below with reference to FIG. 4). The electrodes 22 and 24 contact the patient's skin through the openings 29′, 29 in adhesive 30. Upon depression of push button switch 12, the electronic circuitry on circuit board assembly 18 delivers a predetermined DC current to the electrodes/reservoirs 22, 26 and 24, 28 for a delivery interval of predetermined length, e.g., about 10 minutes. Preferably, the device transmits to the user a visual and/or audible confirmation of the onset of the agent delivery, or bolus, interval by means of LED 14 becoming lit and/or an audible sound signal from, e.g., a “beeper.”

Anodic electrode 22 and/or cathodic electrode 24 can be preferably comprised of silver and/or silver chloride, or any suitable electrically conductive material and reservoirs 26 and 28 can be preferably comprised of polymer hydrogel materials. Electrodes 22, 24 and reservoirs 26, 28 are retained by lower housing 20. For anionic biologically active agents, the cathodic reservoir 28 is the “donor” reservoir, which contains the agent, and the anodic reservoir 26 contains a biocompatible electrolyte. One of skill in the art will recognize that with cationic biologically active agents, the reservoirs are reversed.

The push button switch 12, the electronic circuitry on circuit board assembly 18 and the battery 32 are adhesively “sealed” between upper housing 16 and lower housing 20. Upper housing 16 is preferably composed of rubber or other elastomeric material. Lower housing 20 is preferably composed of a plastic or elastomeric sheet material (e.g., polyethylene) which can be easily molded to form depressions 25, 25′ and cut to form openings 23, 23′. The assembled device 10 is preferably water resistant (i.e., splash proof, and is most preferably waterproof. The system has a low profile that easily conforms to the body thereby allowing freedom of movement at, and around, the wearing site. The anode/agent reservoir 26 and the cathode/salt reservoir 28 are located on the skin-contacting side of device 10 and are sufficiently separated to prevent accidental electrical shorting during normal handling and use.

The device 10 adheres to the patient's body surface (e.g., skin) by means of a peripheral adhesive 30 that has upper side 34 and body-contacting side 36. The adhesive side 36 has adhesive properties which assures that the device 10 remains in place on the body during normal user activity, and yet permits reasonable removal after the predetermined (e.g., 24-hour) wear period. Upper adhesive side 34 adheres to lower housing 20 and retains the electrodes and agent reservoirs within housing depressions 25, 25′ as well as retains lower housing 20 attached to upper housing 16.

The push button switch 12 is located on the top side of device 10 and is easily actuated through clothing. Upon switch activation, a first electric signal configured to facilitate transdermal transport as described herein or a second electric signal configured to facilitate intracellular transport as also described herein can be initiated. Alternatively, the operation can be automated. In one embodiment of electrotransport, an audible alarm signals the start of agent delivery, at which time the circuit supplies a predetermined level of DC current to the electrodes/reservoirs for a predetermined (e.g., 10 minute) delivery interval. The LED 14 remains “on” throughout the delivery interval indicating that the device 10 is in an active agent delivery mode. The battery preferably has sufficient capacity to continuously power the device 10 at the predetermined level of DC current for the entire (e.g., 24 hour) wearing period.

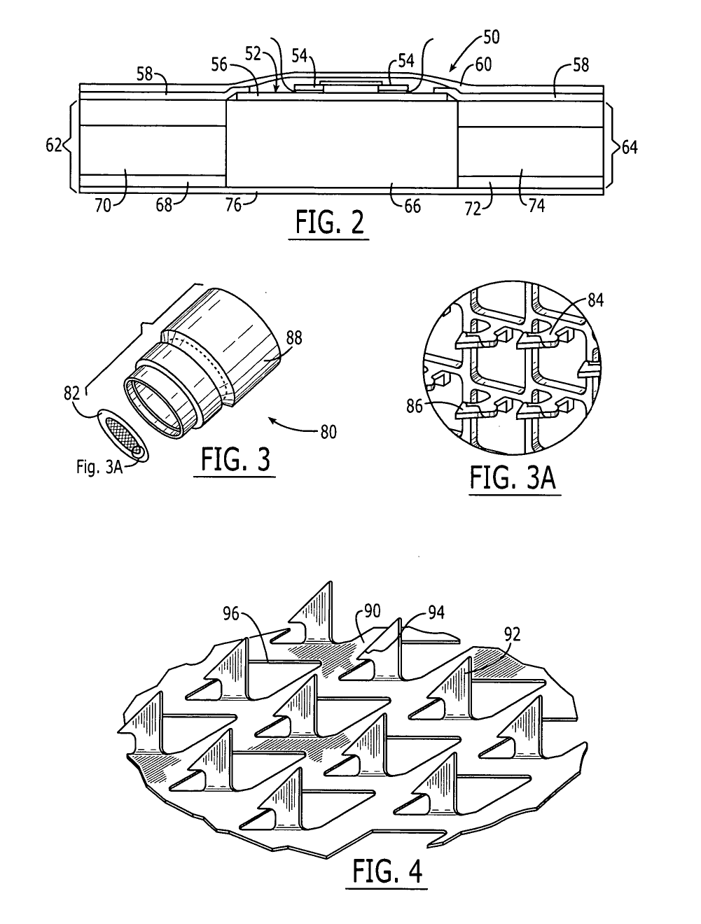

In an alternate embodiment, as shown schematically in FIG. 2, the system of the invention is device 50. Device 50 can have essentially any convenient size or shape, whether square, oval, circular, or tailored for a specific location of the body. Device 50 is flexible and can easily conform to a body (e.g., skin) surface and flex with normal body movement. Device 50 has an electronic circuit 52 having batteries 54 mounted thereon. Generally, circuit 52 is relatively thin and preferably comprised of electronically conductive pathways printed, painted or otherwise deposited on a thin, flexible substrate 56 such as, for example, a film or polymeric web, e.g., circuit 52 is a printed flexible circuit. In addition, to the power source 54, circuit 52 may also include one or more electronic components which control the level, waveform shape, polarity, timing, etc. of the electric current applied by device 50. For example, circuit 52 may contain one or more of the following electronic components: control circuitry such as a current controller (e.g., a resistor or a transistor-based current control circuit), an on/off switch, and/or a microprocessor adapted to control the current output of the power source over time. Circuit 52 has two circuit outputs, each of which is overlain by a layer 58 of an electrically conductive adhesive (ECA). Circuit 52 and ECA layers 58 are preferably covered with a water-impermeable backing layer 60.

Device 50 includes two electrode assemblies indicated by brackets 62 and 64. Electrode assemblies 62 and 64 are separated from one another by an electrical insulator 66, and form therewith a single self-contained unit. For purposes of illustration, the electrode assembly 62 is sometimes referred to as the “donor” electrode assembly while electrode assembly 64 is sometimes referred to as the “counter” electrode assembly. These designations of the electrode assemblies are not critical and may be reversed in any particular device or in operation of the device shown.

In device 50, a donor electrode 68 is positioned adjacent an agent reservoir 70 while a counter electrode 72 is positioned adjacent a return reservoir 74 which contains an electrolyte. Electrodes 68 and/or 72 can comprise microprojection members of the invention, and are formed from any suitable electrically conductive material. Reservoirs 70 and 74 can be polymeric matrices or gel matrices adapted to hold a liquid solvent. Aqueous-based or polar solvents, especially water, are generally preferred when delivering agents across biological membranes such as skin. When using an aqueous-based solvent, the matrix of reservoirs is preferably comprised of a water retaining material and is most preferably comprised of a hydrophilic polymer such as a hydrogel. Natural or synthetic polymer matrices can be employed. Suitable hydrogel formulations are disclosed in Co-Pending Application No. 60/514,433, which is incorporated by reference herein in its entirety.

Insulator 66 is composed of a non-electrical conducting and non-ion-conducting material which prevents current (i.e., current in the form of either electrons or ions) from passing directly between electrode assemblies 62 and 64 and thereby short circuiting the body to which the device is attached. Insulator 66 can be an air gap, a non-ion-conducting polymer or adhesive, or other suitable barrier to ion and electron flow.

The device 50 can be adhered to the skin by means of optional ion-conducting adhesive layer. Alternatively, or in conjunction, the microprojections of the invention may be configured as barbs to anchor the device to the skin. The device 50 also preferably includes a strippable release liner 76 that is removed just prior to application of the device to the skin. Alternatively, device 10 can be adhered to the skin by means of an adhesive overlay of the type that is conventionally used in transdermal agent delivery devices. Generally speaking, an adhesive overlay contacts the skin around the perimeter of the device to maintain contact between reservoirs 24 and 25 and the patient's skin.

In a typical device 50, the agent reservoir 70 contains a neutral, ionized, or ionizable supply of the drug or agent to be delivered and the counter reservoir 74 contains a suitable electrolyte such as, for example, sodium chloride, potassium chloride, or mixtures thereof. Alternatively, device 50 can contain an ionizable, or neutral, supply of agent in both reservoirs 70 and 74 and in that manner both electrode assemblies 62 and 64 would function as donor electrode assemblies. For example, positive agent ions could be delivered through the skin from the anode electrode assembly, while negative agent ions could be introduced from the cathode electrode assembly. Generally, the combined skin-contacting area of electrode assemblies can range from about 1 cm squared to about 200 cm squared, but typically will range from about 5 cm squared to about 50 cm squared.

The agent reservoir 70 and return reservoir 74 of the delivery device 50 must be placed in agent transmitting relation with the patient so as to transdermally deliver the biologically active agent. Usually this means the device is placed in intimate contact with the patient's skin. Various sites on the human body may be selected depending upon the physician's or the patient's preference, the agent delivery regimen or other factors such as cosmetic.

FIG. 3 shows a preferred embodiment of the invention comprising transdermal delivery system 80 that has a microprojection member 82 comprising a plurality of stratum corneum-piercing microprojections 84. FIG. 3A shows a detail view of microprojection member 82 with a biologically active agent 86 coated on the microprojections 84. Preferably, the coating has a thickness of less than about 10 microns. Also preferably, microprojection member 82 is reproducibly and uniformly applied to a patient through the use of an applicator 88, for example a biased (e.g., spring driven) impact applicator. Such devices are discussed in the type described in Trautman et al. U.S. patent application Ser. No. 09/976,673, filed Oct. 12, 2001, the disclosure of which is incorporated herein by reference, can be used to apply the coated microprojection arrays of the present invention. Most preferably, the coated microprojection array is applied with an impact of at least 0.05 joules per cm² of the microprojection array in 10 msec or less.

FIG. 4 shows a partial perspective detail of a microprojection member 90 of the invention. Microprojections 92 form microslits or micropores in the stratum corneum. Optionally, the microprojections 92 can be configured with a barb 94 to help anchor the member on the skin of the patient. Biologically active agents of the invention can pass through openings 96. In drug delivery applications, the agents migrate down the outer surfaces of the microprojections 92 and through the stratum corneum to achieve local or systemic therapy. This movement is assisted using the electrotransport methods of the invention. According to the invention, the number of microprojections 94 and openings 96 of the microprojection array 24 is variable with respect to the desired flux rate, agent being sampled or delivered, delivery device used (i.e., electrotransport, passive, osmotic, pressure-driven, etc.), and other factors as will be evident to one of ordinary skill in the art. In general, the larger the number of microprojections per unit area (i.e., the projection density), the more distributed is the flux of the agent through the skin because there are more pathways.

In one embodiment of the invention, the microprojection density is at least approximately 10 microprojections per cm squared, more preferably, in the range of at least approximately 200-600 microprojections per cm squared. In similar fashion, the number of openings per unit area through which the agent passes is at least approximately 10 openings per cm squared and less than about 1000 openings per cm squared. Similarly, in preferred embodiment, the microprojection piercing elements have a projection length less than 1000 microns. In a further embodiment, the piercing elements have a projection length of less than 500 microns, more preferably, less than 250 microns. The microprojections typically have a width and thickness of about 5 to 50 microns.

Further details of the microprojection member 90 described above and other microprojection devices and arrays that can be employed within the scope of the invention are disclosed in U.S. Pat. Nos. 6,322,808, 6,230,051 B1 and Co-Pending U.S. application Ser. No. 10/045,842, which are incorporated by reference herein in their entirety.

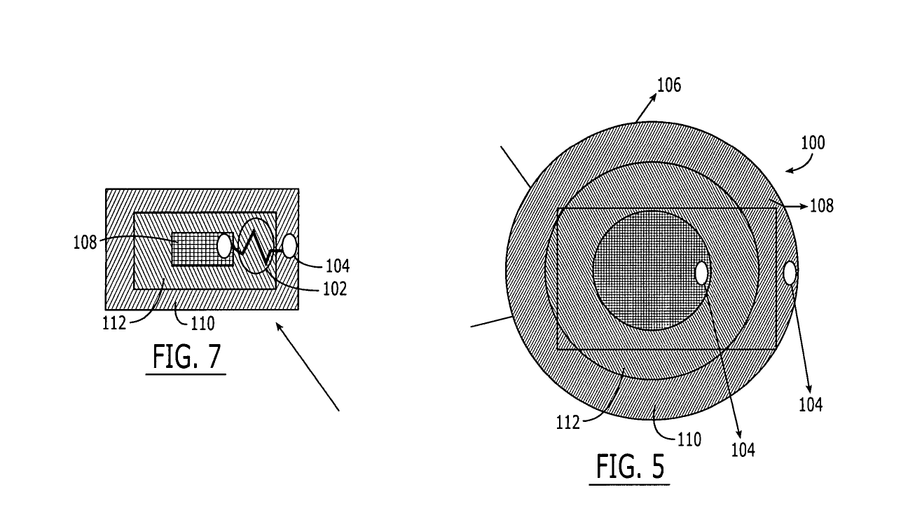

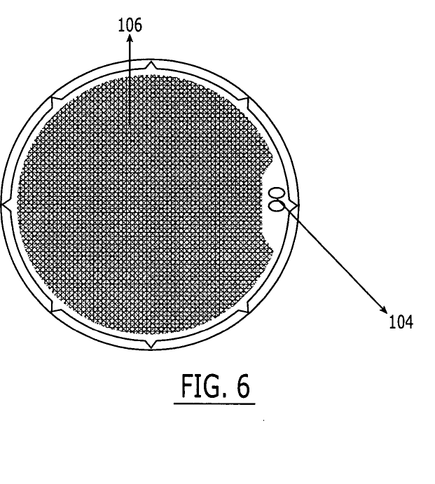

Referring now to FIGS. 5-7, a schematic view of a transdermal delivery system 100 of the invention is shown. System 100 comprises an electric circuit 102 comprising a controller and a source of electrical power, and first and second current conductors 104, which is shown in greater detail in FIG. 6. Microprojection member 106 has a plurality of stratum corneum-piercing microprojections that protrude from said bottom surface of said first microprojection member. Microprojection member 106 has a donor electrode 108 connected to conductor 104 as shown in detail in FIG. 7. A receptor or counter electrode 110 is configured circumferentially around donor electrode 108, and is also connected to circuit 102 by a conductor 104. An insulator 112 prevents shorting between electrodes 108 and 110.

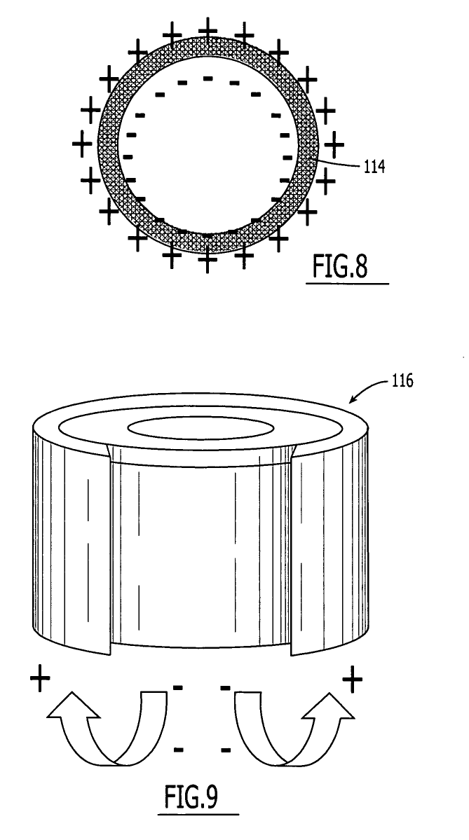

In this embodiment, both the donor electrode 108 and counter electrode 110 comprise a microprojection array. This provides a system having a uniform penetration depth through the stratum corneum. The uniform penetration generates a very homogenous electrical field when voltage is applied across the electrodes. The homogeneity is increased because there is no break at the stratum corneum-electrode interface. Such a homogenous field contributes to the efficiency, reliability and reproducibility of the electrotransport of agents across the skin. One of skill in the art will also recognize that this configuration provides a parallel plate capacitor geometry 114 symmetrically around the circumference of microprojection member 106, as schematically shown in FIG. 8. This configuration maximizes the surface charge density across the insulator interface, which in turn increases the overall electrostatic field. The electrical field 116 shown in FIG. 9 that is generated by this geometry is spherically symmetrical. The configuration also distributes the field over a broad area, maximizing the chance of interaction between the biologically active agent and the field. Further, the use of microprojection arrays facilitates the transport of macromolecules.

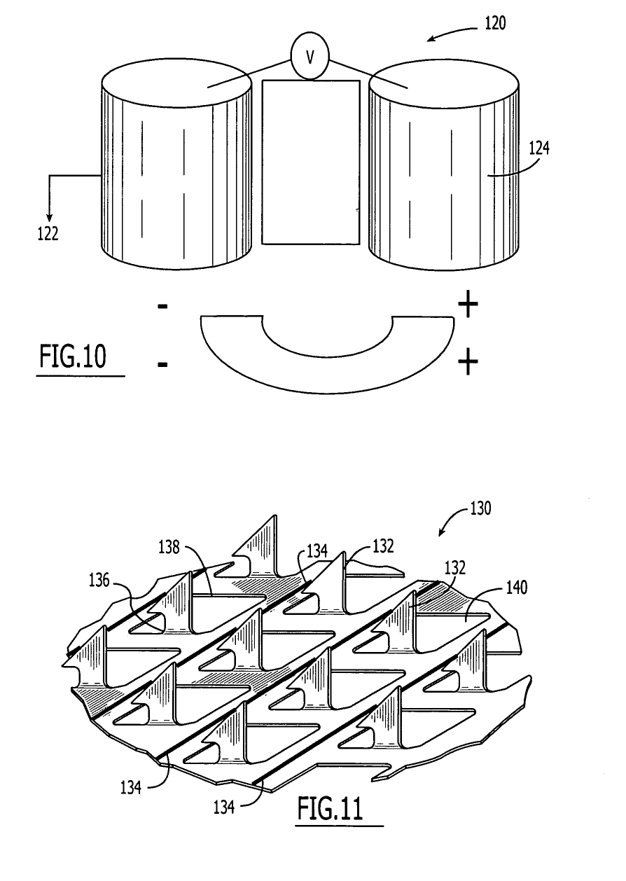

Another embodiment of the invention is shown in FIG. 10. In this configuration, the transdermal delivery device comprises two microprojection electrodes spaced a suitable distance apart. Such a configuration generates semispherical symmetrical electric field 120, comprising a donor electrical field 122 and a counter electrical field 124. As above, using a microprojection array for both electrodes generates a very uniform and homogenous electrical field due to the uniform penetration of the microprojections.

In yet another embodiment of the invention, shown in FIG. 11, interdigitating rows of microprojections form the two electrodes. Specifically, the microprojection member 130 has a plurality of stratum corneum-piercing microprojections 132. Rows of microprojections are electrically isolated by insulator 134 to form donor electrodes 136 and counter electrodes 138. Openings 140 allow the passage of biologically active agent. This configuration also provides the benefit of uniform penetration of both the donor and counter electrodes. Electric discharge between rows of donor electrodes 136 and counter electrodes 138 upon application of an electrical signal can generate electrical fields sufficient to electroporate a cell membrane, thus enhancing intracellular delivery of a biologically active agent.

The embodiments shown in FIGS. 5 and 11, for example, may be conveniently manufactured as two separate units that may then be secured together with an insulating layer between them.

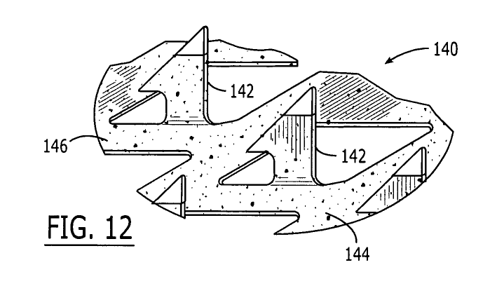

Another aspect of the invention is shown in FIG. 12, which is a partial perspective view of a microprojection member 140. As shown, microprojection member 140 has a plurality of stratum corneum-piercing projections 142. An insulating coating 144 covers the base 146 of microprojection member 140 and the body of microprojections 142. By leaving the tips of the projections bare, electric field densities are highly concentrated at that site. Application of appropriate voltage across the electrodes generates membrane-permeabilizing energies, capable of forming micropores in a cell membrane.

Methods of the invention comprise configuring the control to deliver a first electrical signal to the microprojection member to facilitate electroporation and intracellular electrotransport of the biologically active agent. Preferably, the control is also configured to deliver a second electrical signal to the microprojection member, prior to the first electrical signal, to facilitate transdermal transfer of the biologically active agent.

Electrotransport embodiments of the invention use at least two electrodes that are in electrical contact with some portion of the skin, nails, mucous membrane, or other surface of the body. One electrode, commonly called the “donor” electrode, is the electrode from which the therapeutic agent is delivered into the body. The other electrode, typically termed the “counter” electrode, serves to close the electrical circuit through the body. For example, if the therapeutic agent to be delivered is a positively charged cation, then the anode is the donor electrode, while the cathode is the counter electrode, which serves to complete the circuit. Alternatively, if a therapeutic agent is a negatively charged anion, the cathode is the donor electrode and the anode is the counter electrode. Additionally, both the anode and cathode may be considered donor electrodes if both anionic and cationic therapeutic agent ions, or if uncharged dissolved therapeutic agent, are to be delivered. Furthermore, electrotransport delivery systems generally require at least one reservoir or source of the therapeutic agent to be delivered to the body. Examples of such donor reservoirs include a pouch or cavity, a porous sponge or pad, and a hydrophilic polymer or a gel matrix. Such donor reservoirs are electrically connected to, and positioned between, the anode or cathode and the body surface, to provide a fixed or renewable source of one or more therapeutic agents or drugs.

Electrotransport devices are powered by an electrical power source such as one or more batteries. Typically, at any one time, one pole of the power source is electrically connected to the donor electrode, while the opposite pole is electrically connected to the counter electrode. Since it has been shown that the rate of electrotransport agent delivery is approximately proportional to the electric current applied by the device, many electrotransport devices typically have an electrical controller that controls the voltage and/or current applied through the electrodes, thereby regulating the rate of agent delivery. These control circuits use a variety of electrical components to control the electrical signal, i.e., the amplitude, polarity, timing, waveform shape, etc. of the electric current and/or voltage, supplied by the power source. U.S. Pat. No. 5,047,007, which is hereby incorporated by reference in its entirety, discloses several suitable parameters and characteristics. In embodiments of the invention comprising microprojection member electrodes, it may be desirable to augment the devices with conventional electrotransport electrodes to enhance transdermal delivery.

Electroporation gives temporary access to the interior of the cell by forming micropores and/or otherwise increasing the permeability of the cell membrane. Successful electroporation offers significant benefits such as productions of monoclonal antibodies, cell-cell fusion, cell-tissue fusion, insertion of membrane proteins, and genetic transformation. In addition, the intracellular delivery of dyes and fluorescent molecules using electroporation can benefit research and diagnosis.

Electrodes and electrode arrays can be used to deliver electrical waveforms for therapeutic benefit, including electroporation. Electrical treatment is conducted in a manner that results in a temporary membrane destabilization with minimal cytotoxicity. The intensity of electrical treatment is typically described by the magnitude of the applied electric field. This field is defined as the voltage applied to the electrodes divided by the distance between the electrodes. The electrical signal comprises the pulse magnitude, duration, waveform, and other suitable characteristics. Exemplary pulse magnitude and duration ranges include, but are not intended to be limited to, 1-20,000 volts/cm for a duration in the nanosecond to second range. A preferred range comprises 100-5,000 volts/cm. A particular embodiment comprises a pulse or plurality of pulses in a range of 1-500 volts/cm for a duration in the millisecond range or a pulse or plurality of pulses in a range of 750-1500 volts/cm in the microsecond range. These values are given for example only, and one of skill in the art will be able to select appropriate values based on the intended application. Presently preferred electric field strengths may range from 1000 to 5000 volts/cm for delivering molecules in vivo. Excessive field strength results in lysing of cells, whereas a low field strength results in reduced efficacy. Pulses are usually of the square wave type; however, exponentially decaying pulses may also be used. The duration of each pulse is called pulse width. Electroporation can be performed with pulse widths ranging from microseconds to milliseconds. The number of pulses typically ranges from one to hundred, and preferably, multiple pulses are utilized.

For molecules to be delivered to the cell interior by electroporation, it is important that the molecule of interest be near the exterior of the cell membrane when in the cell is in a permeabilized state. The electrotransport functions of this invention a very suitable for delivery a biologically active agent to the appropriate area prior to electroporation. Accordingly, it is desirable to deliver an electronic signal to the electrodes the will facilitate the transdermal transport of the biologically active agent. The electric signal will be configured to iontophoretically transfer the agent through the patient's skin. Subsequently, an electronic signal configured to electroporate cell membranes may be applied to the electrodes to facilitate the intracellular transport of the biologically active agent. A further enhancement of the invention comprises supplying an additional electronic signal to the electrodes that is configured to transport the agent through the permeabilized cell membrane. As one of skill in the art will appreciate, one or all of the steps can be repeated to control and modify both the electrotransport and electroporation aspects of the invention. Illustrative electrotransport and electroporation agent delivery systems are disclosed in U.S. Pat. Nos. 5,147,296, 5,080,646, 5,169,382 and 5,169383, the disclosures of which are incorporated by reference herein in their entirety.

According to the invention, the coating formulations preferably include at least one wetting agent. As is well known in the art, wetting agents can generally be described as amphiphilic molecules. When a solution containing the wetting agent is applied to a hydrophobic substrate, the hydrophobic groups of the molecule bind to the hydrophobic substrate, while the hydrophilic portion of the molecule stays in contact with water. As a result, the hydrophobic surface of the substrate is not coated with hydrophobic groups of the wetting agent, making it susceptible to wetting by the solvent. Wetting agents include surfactants as well as polymers presenting amphiphillic properties.

In one embodiment of the invention, the coating formulations include at least one surfactant. According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Preferably, the concentration of the surfactant is in the range of approximately 0.001-2 wt. % of the coating solution formulation.

In a further embodiment of the invention, the coating formulations include at least one polymeric material or polymer that has amphiphilic properties. Examples of the noted polymers include, without limitation, cellulose derivatives, such as hydroxyethylcel lulose (HEC), hydroxypropylmethylcellulose (HPMC), hydroxypropylcellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

As will be appreciated by one having ordinary skill in the art, the noted wetting agents can be used separately or in combinations.

According to the invention, the coating formulations can further include a hydrophilic polymer. Preferably the hydrophilic polymer is selected from the following group: poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %, more preferably, in the range of approximately 0.03-10 wt. % of the coating formulation. Even more preferably, the concentration of the wetting agent is in the range of approximately 0.1-5 wt. % of the coating formulation.

According to the invention, the coating formulations can further include a biocompatible carrier, such as those disclosed in Co-Pending U.S. application Ser. No. 10/127,108, which is incorporated by reference herein in its entirety. Examples of suitable biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation. Even more preferably, the concentration of the wetting agent is in the range of approximately 10-40 wt. % of the coating formulation.

According to the invention, the coating formulations can further include a stabilizing agent, such as those disclosed in Co-Pending U.S. Application No. 60/514,533, which is incorporated by reference herein in its entirety. Examples of suitable stabilizing agents include, without limitation, a non-reducing sugar, a polysaccharide, a reducing sugar, or a DNase inhibitor.

The coatings of the invention can further include a vasoconstrictor such as those disclosed in Co-Pending U.S. application Ser. Nos. 10/674,626 and 60/514,433, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulations include at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

According to the invention, the coating formulations can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

Other known formulation additives can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 10. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

In one embodiment, the coating thickness is less than 25 microns, more preferably, less than 10 microns as measured from the microprojection surface. Even more preferably, the coating thickness is in the range of approximately 1 to 10 microns.

In other aspects of the invention, the biologically active agent is contained in a hydrogel formulation. Preferably, the hydrogel formulation(s) contained in a reservoir adjacent one of the electrodes comprise water-based hydrogels, such as the hydrogel formulations disclosed in Co-Pending Application No. 60/514,433, which is incorporated by reference herein in its entirety.

As is well known in the art, hydrogels are macromolecular polymeric networks that are swollen in water. Examples of suitable polymeric networks include, without limitation, hydroxyethylcel lulose (HEC), hydroxypropylmethylcellulose (HPMC), hydroxypropylcellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. These polymers can be obtained in various grades presenting different average molecular weight and therefore exhibit different rheological properties.

According to the invention, the hydrogel formulations also include one surfactant (i.e., wetting agent). According to the invention, the surfactant(s) can be zwitterionic, amphoteric, cationic, anionic, or nonionic. Examples of surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives such as sorbitan laurate, and alkoxylated alcohols such as laureth-4. Most preferred surfactants include Tween 20, Tween 80, and SDS.

Preferably, the hydrogel formulations further include polymeric materials or polymers having amphiphilic properties. Examples of the noted polymers include, without limitation, cellulose derivatives, such as hydroxyethylcellulose (HEC), hydroxypropylmethylcellulose (HPMC), hydroxypropylcellulose (HPC), methylcellulose (MC), hydroxyethylmethylcellulose (HEMC), or ethylhydroxyethylcellulose (EHEC), as well as pluronics.

Preferably, the concentration of the surfactant is comprised between 0.001% and 2 wt. % of the hydrogel formulation. The concentration of the polymer that exhibits amphiphilic properties is preferably in the range of approximately 0.5-40 wt. % of the hydrogel formulation.

As indicated, according to at least one additional embodiment of the invention, the invention, the hydrogel formulations contain at least one biologically active agent, for example, a vaccine. Preferably, the vaccine comprises one of the aforementioned vaccines, including, without limitation, viruses and bacteria, protein-based vaccines, polysaccharide-based vaccine, and nucleic acid-based vaccines.

In a further embodiment of the invention, the hydrogel formulations contain at least one pathway patency modulator, such as those disclosed in Co-Pending U.S. application Ser. No. 09/950,436, which is incorporated by reference herein in its entirety. Suitable pathway patency modulators include, without limitation, osmotic agents (e.g., sodium chloride), zwitterionic compounds (e.g., amino acids), and anti-inflammatory agents, such as betamethasone 21-phosphate disodium salt, triamcinolone acetonide 21disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, and EDTA.

According to the invention, the hydrogel formulations can also include a non-aqueous solvent, such as ethanol, isopropanol, propylene glycol, polyethylene glycol and the like, dyes, pigments, inert fillers, permeation enhancers, excipients, and other conventional components of pharmaceutical products or transdermal devices known in the art.

The hydrogel formulations can further include at least one vasoconstrictor. Suitable vasoconstrictors similarly include, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

### EXAMPLE 1

Electroporation effects of the inventive system were observed using the microprojection array member of the type shown in FIGS. 5-7. The microprojection member comprised an electroporation pulse delivery electrode having a concentric additional microprojection array electrode ring around the core microprojection array. Both arrays are separated by a non-conductive ring, generating two electroporation electrodes, each providing a plurality of microprojections in position where intracellular uptake is desired. In this example, an increase of intracellular DNA uptake after microprojection DNA delivery into HGP was achieved by applying electroporation pulses through the microprojection array electrodes. DNA uptake was monitored by detecting gene expression on the mRNA level and a comparison of the efficacy of this system was made to a conventional, prior art macro-needle array electrode. This example includes seven treatment groups, comprising microprojection arrays with and without electrotransport augmentation and a commercial macro-needle electroporation system.

Group 1: DNA delivery by microprojection array MF S250 without any electrotransport augmentation of intracellular delivery.

Group 2: DNA delivery by microprojection array S250 followed by electroporation applied through a commercially available macro-needle array electrode (Cytopulse, Inc.).

Group 3: DNA delivery by microprojection array S250 followed by electroporation pulses configured for electroporation applied through the concentric microprojection array electrodes.

Group 4: DNA delivery by microprojection array MF 1065 without any electrotransport augmentation of intracellular delivery.

Group 5: DNA delivery by microprojection array MF 1065 followed by electroporation applied through a commercially available macro-needle array electrode (Cytopulse, Inc.).

Group 6: DNA delivery by microprojection array MF 1066 without any electrotransport augmentation of intracellular delivery.

Group 7: DNA delivery by microprojection array MF 1066 followed by electroporation applied through a commercially available macro-needle array electrode (Cytopulse, Inc.).

### Materials and Methods

Microprojection arrays comprising titanium microprojections bent at an angle of approximately 90° to the plane of the sheet, an area of approximately 2 cm² and increasing protrusion length, MF S250 (250 μm), MF 1065 (400 μm), and MF 1066 (600 elm), were used. The arrays were coated with CEN014 (beta-galactosidase expression plasmid) with a loading of 40 μg DNA per array. A closed backing adhesion pad was used to secure the array to the skin. The electrotransport conditions were configured for electroporation (EP) and were 4 EP pulses, 100V/cm, 40 msec., 2 Hz., when delivered by Cytopulse 2×6 needle array electrode (6NA) inserted into the skin at the microprojection array delivery site and 4 EP pulses, 100V/cm, 40 msec., 2 Hz., when delivered by the microprojection array electrode using a BioRad GenePulser Xcell pulse generator.

Delivery of the DNA to the skin of hairless guinea pigs (HGPs) was as follows. Coated microprojection arrays were applied to live HGP for 1 minute and the application site marked. DNA delivery by microprojection array was augmented by electrotransport, as indicated in Table 1. Residual analyses showed an average delivery rate of 48%, or an average delivery into the skin of 19.5 μg DNA. Electroporation (EP) was done immediately following DNA delivery by the microprojection array, while all animals remained under anesthesia.

TABLE 1

| | | | | | Gene |
|---|---|---|---|---|---|
| | | | | Augmentation | Expression |
| Group | n | MF | Electrode type | Method | (rtPCR) |
| 1 | 2 | S250 | none | none | 0/2 positive |
| 2 | 3 | S250 | 6 needle array | EP | 0/3 positive |
| 3 | 3 | S250 | MF micro-needle | EP | 2/3 positive |
| | | | array | | |
| 4 | 2 | 1065 | none | none | 1/2 positive |
| 5 | 3 | 1065 | 6 needle array | EP | 1/3 positive |
| 6 | 2 | 1066 | none | none | 1/2 positive |

Intracellular uptake of plasmid DNA after microprojection array DNA delivery was determined by measuring gene expression of the encoded beta-galactosidase protein on the mRNA level by rtPCR. One day (24 hrs.) after DNA delivery, animals were sacrificed and 8 mm skin biopsies were obtained. Biopsies were obtained from the center of all treatment sites, intradermal injection sites, and untreated skin sites. Biopsies were weighed, homogenized by mincing and short sonication. RNA was extracted using the Stratagen RNA extraction Kit (Absolutely RNA™ RT-PCR Miniprep Kit (Stratagene 400800) according to the manufacturer's protocol, and first strand cDNAs were generated using the ProSTAR First strandRT-PCR kit (Stratagene Cat# 200420). rtPCR reactions were performed using an Invitrogen Kit: PCR Supermix (Invitrogen 10572014).

PCR conditions for this example were as follows. The primers used included an Intron RT 5′ primer-5′CCG GGA ACG GTG CAT TGG AA 3′ [SEQ. ID NO: 1] and a #1057 b-gal intron RT 3′ primer-5′ ATC GGC CTC AGG AAG ATC GC 3′ [SEQ. ID NO: 2]. The fragments provided were 1286 bp (plasmid) or 459 bp (message). 2 μl primers were used with 5 μg total starting RNA in a 50 μl reaction. The PCR reaction conditions were 95° C. for 5 min, 40 cycles of 92° C. for 1 min, 66° C. for 30 sec, 72° C. for 1 min, and a 10 min extension at 72° C. 8 μl of the PCR reaction was analyzed by gel electrophoresis for the presence of a beta-galactosidase mRNA specific fragment of 131 nucleotides. This method detects beta-galactosidase expression in a qualitative manner.

As can be seen in Table 1, when the microprojection array S250 was used to transfer DNA to HGP skin without electrotransport augmentation (Group 1), no expression could be detected (n=2). No expression was detected after electroporation using a commercial six needle array applicator (n=3) either (Group 2). However, delivery of DNA using the microprojection array with integrated concentric electrode and application of an electric field directly after DNA delivery yielded two out of three mRNA positive tissue biopsies, showing that this electrode is suitable for delivering electric discharges and enhancing intracellular DNA uptake and expression in skin. In this experimental group, the microprojection array electrode was superior to the commercial six macro-needle two-row electrode in enhancing gene expression after DNA delivery by microprojection member.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A system for transdermally delivering a biologically active agent, comprising:
a first electrode having top and bottom surfaces and a plurality of stratum corneum-piercing microprojections that protrude from said bottom surface of said first electrode;
a second electrode;
a biologically active agent source associated with said first electrode containing said biologically active agent;
and a circuit adapted to deliver a first electrical signal to said first electrode and said second electrode capable of electroporating a cell membrane.

2. The system of claim 1, wherein said first electrical signal facilitates intracellular transfer of said biologically active agent.

3. The system of claim 1, wherein said first electrical signal is configured to generate electric field densities in the range of approximately 100 V/cm to 5,000 V/cm.

4. The system of claim 2, wherein said circuit is further configured to deliver a second electrical signal to said first electrode and said second electrode that facilitates transdermal transfer of said biologically active agent.

5. The system of claim 1, wherein said second electrode has top and bottom surfaces and a plurality of stratum corneum-piercing microprojections that protrude from said bottom surface of said second electrode.

6. The system of claim 5, wherein said first electrical signal generates a substantially homogenous electrical field.

7. The system of claim 6, wherein said first electrode and said second electrode comprise a first microprojection member.

8. The system of claim 7, wherein said first electrode and said second electrode comprise zones of said first microprojection member and wherein said first electrode and said second electrode are separated by an insulator.

9. The system of claim 8, wherein said first electrode comprises a circular zone and said second electrode comprises a circumferential zone around said circular zone.

10. The system of claim 9, wherein said first electrical signal generates a spherically symmetrical electric field.

11. The system of claim 9, wherein said first electrode and said second electrode comprise a parallel plate capacitor geometry around a circumference of said microprojection member.

12. The system of claim 7, wherein said first electrode and said second electrode comprise alternating rows of said stratum corneum-piercing microprojections separated by an insulator.

13. The system of claim 6, wherein said first electrode comprises a first microprojection member and wherein said second electrode comprises a second microprojection member.

14. The system of claim 13, wherein said first microprojection member and said second microprojection member are positioned to generate a semispherically symmetrical electrical field.

15. The system of claim 5, further comprising an insulating coating disposed on said first microprojection member configured to maximize electric field densities to electroporate cells.

16. The system of claim 1, wherein said biologically active agent comprises an immunologically active agent.

17. The system of claim 1, wherein said biologically active agent is selected from the group consisting of anti-infectives, antibiotics, antiviral agents, analgesics, fentanyl, sufentanil, remifentanil, buprenorphine, analgesic combinations, anesthetics, anorexics, antiarthritics, antiasthmatic agents, terbutaline, anticonvulsants, antidepressants, antidiabetic agents, antidiarrheals, antihistamines, anti-inflammatory agents, antimigraine preparations, antimotion sickness preparations such as scopolamine and ondansetron, antinauseants, antineoplastics, antiparkinsonian drugs, antipruritics, antipsychotics, antipyretics, antispasmodics, anticholinergics, sympathomimetrics, xanthine derivatives, cardiovascular preparations, calcium channel blockers, nifedipine, beta blockers, beta-agonists, dobutamine, ritodrine, antiarrythmics, antihypertensives, atenolol, ACE inhibitors, ranitidine, diuretics, vasodilators, central nervous system stimulants, cough and cold preparations, decongestants, diagnostics, hormones, parathyroid hormone, hypnotics, immunosuppressants, muscle relaxants, parasympatholytics, parasympathomimetrics, prostaglandins, proteins, peptides, psychostimulants, sedatives and tranquilizers.

18. The system of claim 1, wherein said biologically active agent source comprises a biocompatible coating on said microprojections.

19. The system of claim 18, wherein said coating further comprises a compound selected from the group consisting of a surfactant, an amphiphilic polymer, a hydrophilic polymer, a biocompatible carrier, a stabilizing agent, a vasoconstrictor, and a pathway patency modulator.

20. The system of claim 1, wherein said biologically active agent source comprises a hydrogel.

21. The system of claim 20, wherein said hydrogel further comprises a compound selected from the group consisting of a macromolecular polymer network, a surfactant, an amphiphilic polymer, a vasoconstrictor, and a pathway patency modulator.

22. A method for delivering a biologically active agent comprising the steps of:
a) providing a transdermal delivery system comprising:
i) a first electrode having top and bottom surfaces and a plurality of stratum corneum-piercing microprojections that protrude from the bottom surface of the first electrode;
ii) a second electrode;
iii) a biologically active agent source associated with the first electrode containing a biologically active agent; and
iv) a circuit adapted to deliver a first electrical signal to the first and second electrodes capable of electroporating a cell membrane; and
b) delivering said first electrical signal to the first electrode and the second electrode to facilitate intracellular transport of the biologically active agent.

23. The method of claim 22, wherein the step of delivering said first signal generates electric field densities in the range of approximately 100 V/cm to 5,000 V/cm.

24. The method of claim 22, further comprising the step of repeatedly delivering said first electrical signal.

25. The method of claim 22, further comprising the step of delivering a second electrical signal to said first electrode and said second electrode that facilitates transdermal transfer of said biologically active agent, wherein delivering said second electrical signal occurs before delivering said first electrical signal.

26. The method of claim 22, wherein said second electrode has top and bottom surfaces and a plurality of stratum corneum-piercing microprojections that protrude from the bottom surface of said second electrode and wherein the step of delivering a first electrical signal further comprises generating a substantially homogenous electric field.

27. The method of claim 26, wherein said first electrode and said second electrode comprise a first microprojection member.

28. The method of claim 27, wherein said first electrode comprises a circular zone of said microprojection member and said second electrode comprises a circumferential zone around said circular zone and wherein the step of delivering said first electrical signal generates a spherically symmetrical electric field.

29. The method of claim 27, wherein said first electrode and said second electrode comprise alternating rows of said stratum corneum-piercing microprojections on said first microprojection member and wherein said alternating rows are separated by an insulator.

30. The method of claim 26, wherein said first electrode comprises a first microprojection member and said second electrode comprises a second microprojection member and wherein said first microprojection member and said second microprojection member are positioned so that delivering said first electrical signal generates a semispherically symmetrical electrical field.

31. The method of claim 26, further comprising the step of disposing an insulating coating on said microprojections to maximize electric field densities to electroporate cells.

32. The method of claim 31, wherein the step of disposing an insulating coating on said microprojections comprises leaving tips of said microprojections uncoated.

33. The method of claim 25, further comprising the step of delivering a third electrical signal to said first electrode and said second electrode to transport said biologically active agent across said cell membrane after the steps of delivering said second electrical signal and delivering said first electrical signal.
